(19) United States
(12) Patent Application Publication
(10) Pub. No.: US 2010/0092499 A1
(43) Pub. Date: Apr. 15, 2010

(54) Alpha Thymosin Peptides as Cancer Vaccine Adjuvants

(75) Inventors: Gustavo Antonio Moviglia, Buenos Aires (AR); Alfred R. Rudolph, Los Altos Hills, CA (US)

Correspondence Address: ROTHWELL, FIGG, ERNST & MANBECK, P.C., 1425 K STREET, N.W., SUITE 800, WASHINGTON, DC, 20005, US

(73) Assignee: SCICLONE PHARMACEUTICALS, INC., San Mateo, CA (US)

(21) Appl. No.: 11/720,909

(22) PCT Filed: Dec. 6, 2005
(86) PCT No.: PCT/US05/43985

§ 371 Date: Dec. 29, 2009

Related U.S. Application Data
(60) Provisional application No. 60/633,175, filed on Dec. 6, 2004.

(51) Int. Cl. A61K 39/00; A61P 35/00

(52) U.S. Cl. 424/185.1

(57) ABSTRACT

A pharmaceutical combination and method for enhancing cancer vaccine effectiveness in a subject, utilize an immune response-triggering cancer vaccine capable of eliciting an immune system response in a subject; and a vaccine effectiveness-enhancing amount of an alpha thymosin peptide, which enhances the immune system response in the subject, wherein the cancer vaccine and the alpha thymosin peptide can be administered separately or together.

### CROSS-REFERENCE TO RELATED APPLICATION

This application claims benefit from U.S. Provisional Application Ser. No. 60/633,175, filed Dec. 6, 2004.

### FIELD OF THE INVENTION

The present invention relates to the field of cancer treatment.

### BACKGROUND OF THE INVENTION

Cancer is a leading cause of death throughout the world. Non specific approaches to cancer treatment like surgery, chemotherapy and radiotherapy have been successful in selective groups of patients. Immunotherapy constitutes a new area for the treatment of cancer. The general principle is to provide to the treated subject the ability to increase the immunology activity against the tumor cells. There are a number of strategies that have emerged during the last few years and are currently under development. These strategies involve: transfer of allogenic lymphocytes, infra tumor implantation of immune reactive cells, systemic vaccination to generate a tumor specific immune response and others.

There remains a need in the art for improved anti-cancer treatments and compositions.

### SUMMARY OF THE INVENTION

In accordance with the present invention, a pharmaceutical combination and method for enhancing cancer vaccine effectiveness in a subject, utilize an immune response-triggering cancer vaccine capable of eliciting an immune system response in the subject, and a vaccine effectiveness-enhancing amount of an alpha thymosin peptide, which enhances said immune system response in said subject; wherein said cancer vaccine and said alpha thymosin peptide can be administered separately or together.

### DESCRIPTION OF THE PREFERRED EMBODIMENTS

The present invention is directed to treatment of tumors and cancers in a subject, preferably a mammalian subject, and most preferably a human subject.

Advanced cancer is resistant to usual cancer treatment methods. Some cancer Vaccines have shown some activity in reducing or stopping the disease progression, associated or not with tumor response, and increasing survival. Administration of an alpha thymosin peptide such as thymalfasin (thymosin alpha-1) has a positive adjuvant effect on vaccine treatment of cancer patients, both reducing the tumor size and increasing the survival rate on advanced cancer patients, including those who did not respond to cancer vaccine alone (e.g., dendritic cell immunization).

This invention is related to the treatment of cancers and tumors. In one embodiment, the invention is directed to the immunostimulation activity of an alpha thymosin peptide such as the immunomodulator substance thymalfasin in the treatment of patients with neoplastic disease such as cancer, including, but not limited to, breast cancer, that received a treatment with an oncology vaccine. This includes the improvement of the therapeutic response due the addition of an alpha thymosin peptide in patients treated with cancer vaccines like dendritic cell vaccines, but is not exclusive for this kind of cancer vaccine.

The present invention is exemplified in the treatment of breast cancer. However, cancers which may be treated using the present invention may include but are not limited to primary melanoma, metastatic melanoma, adenocarcinoma, squamous cell carcinoma, adenosquamous cell carcinoma, thymoma, lymphoma, sarcoma, lung cancer, liver cancer, non-Hodgkins lymphoma, Hodgkins lymphoma, leukemias, uterine cancer, prostate cancer, ovarian cancer, pancreatic cancer, colon cancer, multiple myeloma, neuroblastoma, NPC, bladder cancer, cervical cancer, kidney cancer, brain cancer, bone cancer, uterine cancer, stomach cancer, rectal cancer, and the like.

Alpha thymosin peptides comprise thymosin alpha 1 (TA1) peptides including naturally occurring TA1 as well as synthetic TA1 and recombinant TA1 having the amino acid sequence of naturally occurring TA1, amino acid sequences substantially similar thereto, or an abbreviated sequence form thereof, and their biologically active analogs having substituted, deleted, elongated, replaced, or otherwise modified sequences which possess bioactivity substantially similar to that of TA1, e.g., a TA1 derived peptide having sufficient amino acid homology with TA1 such that it functions in substantially the same way with substantially the same activity as TA1. Suitable dosages of the alpha thymosin peptide can be in the range of about 0.001-10 mg/kg/day.

The terms “thymosin alpha 1” and “TA1” refer to peptides having the amino acid sequence disclosed in U.S. Pat. No. 4,079,137, the disclosure of which is incorporated herein by reference.

Effective amounts of an alpha thymosin peptide are cancer vaccine-enhancing amounts which may be dosage units within a range corresponding to about 0.1-20 mg of TA1, preferably 0.5-10 mg of TA1. More preferably, the dosage unit comprises about 1-4 mg of TA1. Most preferably, the dosage unit comprises about 1.6-3.2 mg of TA1.

Thymosin alpha 1 (TA1), initially isolated from Thymosin Fraction 5 (TF5), has been sequenced and chemically synthesized. TA1 is a 28 amino acid peptide with a molecular weight of 3108.

Cancer vaccines for use in accordance with preferred embodiments of the invention are dendritic cell vaccines.

Cancer vaccines can be administered to a subject in accordance with the present invention in any defective dosages. Such dosages may fall in the range of from about 1×10⁻⁹ g to about 1×10⁻³ g. In other embodiments, suitable effective cancer vaccine dosages may be within the range of about 1×10⁻⁸ g to about 1×10⁻⁴ g. The cancer vaccine may be administered to the subject in any effective number of doses e.g., between 1-20 or more doses. Preferably, the cancer vaccine is administered in a plurality of doses, e.g., from about 2 to about 15 doses, more preferably from about 4-10 doses, and most preferably about 6 doses. In particularly preferred embodiments, the vaccine is administered to healthy lymph nodes of the subject once about every 3 weeks during a course of administration.

In preferred embodiments, an immune response-triggering cancer vaccine is administered to a subject in conjunction with administering an alpha thymosin peptide to the subject, wherein the vaccine and the alpha thymosin peptide are administered to the subject separately and/or together. In one embodiment, the alpha thymosin peptide is administered substantially concurrently with administration of the vaccine, at least during one administration of the vaccine. In preferred embodiments, both the vaccine and the alpha thymosin peptide are administered by injection. Preferably, both the vaccine and the alpha thymosin peptide are administered to the subject a plurality of times. In preferred embodiments, the alpha thymosin peptide is administered twice weekly during a course of administration. It is particularly preferred that a course of administration last about six months. In one embodiment, the invention is applicable to treatment of cancer in subjects who are non-responders to cancer vaccine treatment alone.

In particularly preferred embodiments, an alpha thymosin peptide is administered by subcutaneous injection twice weekly in pharmaceutical dosage units within a range of about 1-4 mg (e.g., about 1.6-3.2 mg) in conjunction with administration to the subject of the cancer vaccine. However, it is to be understood that pharmaceutical dosage units containing an alpha thymosin peptide and/or a cancer vaccine may be formulated in any suitable manner for administration by any suitable route.

According to one aspect of this embodiment of the present invention, the dosage unit comprising an alpha thymosin peptide is administered to the subject on a routine basis. For example, the dosage unit can be administered more than once daily, once daily, weekly, monthly, etc. The dosage unit may be administered on a bi-weekly basis, i.e., twice a week, for example, every third day. The dosage unit of alpha thymosin peptide may also be administered on a thrice weekly basis, i.e., three times per week.

Administration of the alpha thymosin peptide and vaccine may take place by any suitable means, such as injection, infusion or orally. In particularly preferred embodiments, administration is by injection.

When a vaccine and the alpha thymosin peptide are administered concurrently, they can be provided as a single composition including the vaccine and the alpha thymosin peptide.

Compositions including a vaccine and/or the alpha thymosin peptide can also include one or more pharmaceutically acceptable carriers and optionally other therapeutic ingredients. Formulations suitable for injection or infusion include aqueous and non-aqueous sterile injection solutions which may optionally contain antioxidants, buffers, bacteriostats and solutes which render the formulations isotonic with the blood of the intended recipient, and aqueous and non-aqueous sterile suspensions which may include suspending agents and thickening agents. The formulations may be presented in unit-dose or multi-dose containers, for example, sealed ampules and viles, and may be stored in a freeze-dried (lyophilized) condition requiring only the addition of the sterile liquid carrier, for example, water for injection, immediately prior to use.

Advanced cancer is resistant to the usual cancer treatment methods. Some cancer vaccines have shown activity to reduce or stop the disease progression and increase survival. The administration of an alpha thymosin peptide such as thymalfasin (thymosin alpha-1) has a positive effect on vaccine treatment both reducing the tumor size and increasing the survival rate, including in advanced cancer patients who did not respond to a cancer vaccine alone (such as dendritic cell immunization).

There are three systems in charge of keeping the homeostasis in the human body: the immune, the endocrine and the nervous systems. The immune system is in charge of favoring the cell and tissue repair and differentiation, as well as to preserve their identity by keeping their inner and external environments. Therefore, the two main functions of the immune system are the regulatory function and the effector function. Both of these functions are performed by the same cell population in dynamic response to the needs of the organism.

The immune system plays an active role in cancer therapy and can prevent organ dysfunction and the appearance of a neoplasm.

From a therapeutic point of view, Immunotherapy in cancer means essentially the stimulation of the immune system through a variety of reagents such as vaccines, T cell infusions, or cytokines. These reagents can act through several mechanisms of action:
1) by stimulating the anti-tumor response;
2) by decreasing suppressor mechanisms;
3) by altering tumor cells to increase their immunogenicity and make them more susceptible to immunologic defenses;
4) by improving tolerance to cytotoxic drugs or radiotherapy.

Cancer is caused by a variety of genetic defects that occur in genes that encode for proteins involved in cell growth. The components of the immune system, antibodies and T cells, are ineffective to recognize or respond to defective genes, but they do recognize and respond to the abnormal proteins the cancer-causing genes encode. The immune system may attack cancer through B and T lymphocytes.

Antibodies are proteins produced by B cells in response to a foreign substance. Each antibody binds to a specific antigen. The major protective effects of antibodies take place through the amplifying effects of the “complement” system, a collection of about 20 different proteins. When an antibody binds with an antigen, a specific reactive site on the antibody is activated. This site binds with a molecule of the complement system and sets a cascade of reactions. Opsonization and phagocytosis are among the more important complement effects. They strongly activate phagocytosis by neutrophils and macrophages. This type of antibody-mediated effect is known as antibody-dependent cell mediated cytotoxicity (ADCC). ADCC has the advantage of catalyzing T-cell activity, as the digested foreign cell proteins are presented on the major histocompatibility complex (MHC) molecules of the antigen-presenting cell (APC) as peptides. Antibodies have also been shown to kill cells by blocking growth mechanisms, particularly in cancer cells.

Cytotoxic T cells (CD8+)-cells are specific for class I MHC molecules and-react to peptide antigens expressed on the surface of a cell once they are presented as protein or peptides fragments—displayed in the MHC. The peptide and the MHC together activatettract T cells. This T cell destroys the carrier cell by perforating its membrane with enzymes or by triggering an apoptotic or self-destructive pathway, destroying these invasive cells.

The helper T cells (CD4+) are the regulators of the immune system activities. The CD4+ T cells also recognize the class II MHC. CD4+ T cells increase the immune response by secreting cytokines (like interleukin-2-(IL2)) that stimulate either a cytotoxic T cell response (T-helper 1) or an antibody response (T-helper 2). These cytokines stimulate B cells to produce antibodies, or enhance CD8+ T cell production. CD4+ T cells form a series of protein mediators called cytokines, which act on other cells of the immune system, enhancing the action of the entire immune system's response.

The genetic alterations of cancer cells (oncocytes) cause the appearance of molecules different from those of a non altered adult cell. These different molecules, called tumor antigens or tumor associated antigens; are the target of the effector reaction.

At the same time, the oncocyte generates cytokines to induce its own DNA replication and its own differentiation processes, for example Interferon β that is secreted by virally infected cells stops the viral replication on neighboring cells.

Other cytokines, such as IL6 and Transforming Growth Factor β (TGFβ), unsuccessfully seek repairaration of the genetic damage; although they induce cell differentiation, they inhibit the action of the Th1 effector immune system.

The toxic effect which starts the cell transformation process can damage the immune protection ability (Immune Surveillance) inducing gene mutation and immunosuppression. Moreover, the new oncocyte, trying unsuccessfully to repair its altered DNA, increases the production of TGFβ and or related cytokines inducing immune tolerance to it, so the genetically altered cells originate a neoplasm.

Last investigations have shown that tumors are immunogenic and it is possible that they produce long-term immunologic memory. Another important point is the relapse of the tumor modifying the long-term survival of cancer patients. Some patients can initially respond to such usual therapy as chemotherapy, surgery, or radiation, but the tumor may recur. It is known that patients who have undergone renal transplantation have an estimated 3 to 5 times higher overall incidence of cancer in long-term follow-up than the general population; that may be due in part to their long-term immunosuppression.

Antigens are foreign substances recognized for their destruction by the cells of the immune system. When cells become cancerous, they produce new, unfamiliar antigens. The immune system may recognize these antigens as foreign, and contain or even destroy the cancer cells. Viral proteins—hepatitis B virus (HBV), Epstein-Barr virus (EBV) and human papillomavirus (HPV)—are important in the development of hepatocellular carcinoma, lymphoma, and cervical cancer, respectively. Oncogenic proteins, glycosylated proteins, and carbohydrates are tumor antigens. Many of these proteins are shared between multiple tumor types, and more than 500 tumor antigens have been defined.

The immune response of the body does not appear to be robust enough in patients with cancer. Proteins expressed by cancers can elicit an immune response.

### Vaccination

There are many reasons why there is an inefficient immune response. The cytokine environment does not allow amplification of CD4+ T cells. As tumors grow, they can secrete immunosuppressive factors—either by directly modulating the immune response, such as viral proteins binding to immune receptor molecules and preventing them from being exposed on the surface of a virally infected cell; or even by secreted factors by the tumor itself that downregulate immune system activation.

Immunotolerance is a major mechanism by which tumors escape immune evasion. The design of immunotherapeutic strategies can be effective to eradicate cancer cells. They are focused on making “self” more immunogenic by using immune system activators, supplying antigen-presenting cells, or actually predigesting some of these tumor antigen proteins into immunogenic peptides.

A clinically useful tumor vaccine must immunize against multiple proteins, targeting the important proteins involved in malignant transformation. In this way, the use of drugs or substances called immunomodulators can increase or modify the natural immune response improving the histological and clinical results of the vaccination act. A successful immunotherapy should be focused on the handling the regulatory activities of the immune system to destroy cancer cells and prevent its recurrence.

In a preferred embodiment, the present invention focuses on both of the above described immune activities. Alive and modified autologous tumor cells are used to boost autologous naive Dendritic Cells (DC). The co-culture of both types of cells is developed in a particular tissue culture media to differentiate naïve DC to effector reaction inducer DC. These DC are injected into a healthy lymph node to start the T cell effector reaction against the patient's tumor cells.

This approach is safe, produces minor toxicity to the patient as well as important and sustained antitumor activity against advanced and well-established tumors.

The following first describes the tumor antigens and cells involved in the Immune Surveillance as well as the counterpart tumor escape forms.

Next are described the main immunotherapy strategies used at present. Then are described the inventive therapeutic approach, its principles, possible action mechanisms, and its advantages as compared with other approaches.

Tumor Antigens (TA): The relevant tumor antigens can be divided into two main categories. The first category involves specific tumor antigens (STA) those found exclusively in the tumor cells, which represent an ideal target for an immune attack.

The second category involves tumor associated antigens (TAA), those found in tumor cells but also in some normal cells in which the quantitative and qualitative expression of their molecules enable their use as to distinguish tumor cells from normal cells.

The purpose of tumor immunotherapy is to treat cancer efficiently through the control and enhancement of the immune response against the STA and TAA. The spontaneous remission observed in some cases of malignant melanomas and renal cell carcinoma is evidence of the accomplishment of this goal

Tumor Specific Antigens (TSA): These antigens can only be detected in oncocytes. These antigens have been identified in tumors from experiment animals as well as in human proteins from a viral origin, mutated oncogenes and proteins related to malignant phenotypes, spontaneous mutations probably caused by genomic instability, characteristic of malignant cells.

The elucidation of the pathways for the antigen presentation through the major histocompatibility complex (MHC) to T cells explains that not only altered cell membrane proteins can be detected as antigens but also inner or internalized proteins may become specific tumor antigens. T cells recognize small peptides which derive from the cellular degradation of cytosolic proteins and are inserted in the peptide cleft of the MHC molecule. These peptides are, together with MHC molecule, later transported to the cell surface. Therefore, any abnormal cell protein is a potential immune agent, not only those proteins detected in the membrane. Then a non functional protein in a tumor cell produced my a mutating allele, as in p53, is potentially a specific tumor antigen

Tumor Associated Antigens (TAA): The TAA are tumor cells molecules that can be expressed by some normal cell at particular differentiation stages. Its quantitative or combined expressions in relation with other cell line or differentiation markers, or a combination of both can be useful for the identification of the transformed cells. The best characterized TAA are the oncofetal antigens, which are expressed during embryogenesis, but absent or almost undetectable in normal adult tissue. The prototype TAA is the carcinoembryonic antigen (CEA). α-fetoprotein and MAGE protein family are included in this kind of antigens.

### Immune Surveillance

A genetically transformed cell presents antigenic proteins different in their quality or quantity from the proteins in normal cells (respectively STA and TAA). The cells and humoral components acting in both innate and adaptative immune response play a role in the destruction response of the transformed cell and the tumor once it has been constituted.

The cells involved in the Immune Surveillance process are:

Natural Killer Cells (NK): they recognize and destroy MHC deprived cells. These cells perform their function through the formation of pores into the membrane of the target cell. These pores are made up of the self-assembly of perforine molecules in the plasmatic membrane. The structure of these cells is somewhat homological to complement C9 and its collocation generates a pore through which cytolytic enzymes of the granzyme type can easily pass. The activation of receptors Fas and TNF α over the tumor cell surface constitutes a second mechanism. Both of these phenomena activate apoptosis. These cytolytic activities caused by the absence of MHC molecules are activities corresponding to innate immune response. On the other hand, the natural killer cells also cooperate in the activity of antibodies directed against tumor. These cells adhere to the tumor cell surface through their Fc receptors and cause the above mentioned lytic phenomena (perforine, Fas activation, TNF α attack). This activity is considered part of the adaptative immune response to tumors.

Due to these functions, the natural killer cells become the main responsible cause of destruction of virally induced tumor cells and small tumors in their onset and they are activated by the action of interferons and interleukin 2. These leukins potentiate the lytic activities of NK Cells. NK cells are said to be activated (LAK, Leukin Activated Killers).

The oncocyte destruction as an innate NK response is inhibited by the even imperceptible presence of MHC1 membrane proteins. However, this presence does not inhibit NK response when this is due to the lytic activity of tumor directed antibodies.

Phagocytic cells: The cells with phagocytic activity possess specific anti tumor action mechanisms that can be used with therapeutic purposes. When activated by T lymphocytes, these cells can transfer into the tumor cell: lysozymes, superoxide radicals, nitric oxide, and TNF, which destroy the tumor cells through different mechanisms. However, their most important anti tumor activity is exercised through their antigen presenting capacity, mainly by their CD4 lymphocytes presenting capacity. It is known that the tumors do. not have MHC2 molecules on their surface; therefore they cannot show their characteristic tumor antigens to the helper cells. The activated macrophages can perform this antigen presentation and induce the activation of both regulatory and effector CD4+ lymphocytes. They also present antigens to CD8+ and B cells.

The most skillful cells in the immune system, as regards their phagocytic and cell presenting features are the dendritic cells. A unique dendritic cell can contact up to 1000 naïve CD4 lymphocytes and because of this, the Dendritic Cells are considered to be the most powerful in the organism. Due to this they have been used with therapeutic purposes. They are currently considered the best adjuvant since their stimulus in an artificially controlled medium induces stimulation of any immune system against the tumor. These cells are also the target of the tumor cells inhibitory secretions. The prostaglandine, TGFβ and IL10 secretions of the tumor have a negative effect over the macrophages by inducing the generation of inhibitory (and regulatory) lymphatic population characteristic of rejection.

Lymphocytes: the most powerful antitumor role is played by T lymphocytes of effector group CD4 and CD8. The appearance and development of their suppressor T cell populations unfortunately enable tumor growth and its metastatic spread all over the body. These suppressor lymphocytes have been characterized as a subpopulation of CD 4 lymphocytes having a CD25 positive marker in their cell membrane. The effector response of T cells directly kills tumor cells and activates the rest of the immune system components. The antitumor immunity directed against CD4 and CD8 populations is antigen specific. These lymphocytes have been detected not only in the patients' peripheral blood, but also in the tumor infiltrating cells. As previously described, CD4 cell activity is the most important as regards quantity and quality of antitumor response. However, its action depends on the antigen presentation performed by the corresponding specialized cell, since tumors do not express MHC II molecules. On the contrary, cytotoxic T cells can recognize cell antigens in the MHC I. However, in regular conditions and due to their lack of co-stimulating molecules, tumor cells induce the anergy of CD8 cells specific against tumors, As opposite, activated CD8 lymphocytes do not require these co-stimulating signals for tumor lysis. The lysis mechanisms they use are similar to those used by the NK cells: apoptosis and pore formation in the plasmatic membrane.

B cells: The potential function of the recipient's response to tumor immunity used to be suggested by the occasional detection of reactive antitumor antibodies in the patient's serum. The fundamental action mechanism is cell lysis through antibodies (ADCC). The antibacterial destruction mechanism helped by complement seems to play a lesser role in the antitumor fight. Finally, several experiments support the idea that a specific antibody attack on the tumor leads to the disappearance of the immune response promoting antigen; thus generating (by negative selection) populations resistant to this lytic mechanism. It is clear, though, that cells become sensitive to their destruction by NK cells if antibodies generate disappearance of the MHC I complexes in the cell membrane. Several monoclonal antibodies, such as herceptin against the protein of oncogene HER2-NEU have been developed for its therapeutic use and commercial sale. This molecule is expressed in 25% of the cells of ovarian and breast metastases and the FDA has approved its therapeutic use for the treatment of patients suffering from this condition. The second of these antibodies is rituximad, which is directed against CD20 cell determinant and it is for this reason that it is being successfully used for the treatment of B lymphomas. Other antibodies are currently under clinical development.

Tumor Cell Immunology: tumor cells present several molecules, which could be the target of an inflammatory antitumor response. However, although the lymphocytes that can recognize these antigens have been isolated in blood adjacent to the tumor, these are unable to generate an efficient effector function against the neoplasm. The cytological characteristics of tumor cells explain or attempt an explanation of this phenomenon: the tumor cells do not have MHCII complexes on their surfaces and that is the reason why they cannot present their ATP to the CD4 lymphocytes and they possess a poor MHC I complex expression.

These properties produce inhibition of NK cell activity, and a poor activation response in CD8 cells. This last phenomenon is aggravated by that most tumor cells do not present co-stimulating molecules on their surface. This lack of receptors for the co-stimulating molecules causes the development of anergic CD8 lymphocytes.

Tumor cells highly secrete anti-inflammatory substances. Some of these substances have not been identified yet. The prostaglandine production acts by blocking the activation of macrophages. This substance can be inhibited by the concomitant administration of indometacine or COX2 inhibitors. Tumor cells can also produce a great amount of TGFβ and IL 10. These cytokines are molecules that control cell differentiation. Since tumor cells lack an adequate cell differentiation, they also lack negative regulation signals so as to control production of the synthesis of this substance. There are studies that show the parallel between the metastatic potentials of pancreatic tumors, breast tumors, gliomas, SCLC and others; and the synthesis of these cytokines. Their most important action is to condition the antigen presenting cells so that they induce the appearance of specific suppressor lymphocytes against tumor antigens.

The dynamic relationship between Immune system and tumor: The techniques for the mix culture of tumor and tumor cells have enabled the detailed study of the antigenic composition of cytotoxic T cells reacting against the melanoma peptides. These have been cloned and used to characterize specific tumor antigens through an amino acid sequence. There were three important findings in these studies. The first one: melanomas have at least five different antigens which can be recognized as cytotoxic T cells. The second finding was the fact that cytotoxic T lymphocytes reacting against melanoma antigens do not expand in vivo. This suggests that the before mentioned antigens are not immunogenic when in vivo. The third finding was the possibility of the negative selection in vitro and possibly also in vivo of the expression of these antigens due to the presence of specific cytotoxic T cells. These findings offer hope for a tumor immunotherapy. At the same time, the findings reveal that these antigens are not highly immunogenic in a natural form and they warn about the possibility of selecting tumor cells in vivo, which could not be recognized and eliminated by cytotoxic T cells.

In order to be able to grow, a tumor must generate a series of dynamic escape mechanisms. If confronted with any antitumor strategy, the tumor responds by its own adaptation through the development of a new escape form.

The detection of abnormal molecules generates primary humoral immunity responses through the appearance of specific antibodies and its subsequent destruction by ADCC. The NK and polymorphonuclear cells take an active part in this phenomenon. This induces a selection in the population of those cells with a low or even inexistent expression of the pertinent surface antigens. At the same time, the phagocytosis of the destroyed cells induces a deferred cell immunity response against those intracellular antigens which may be presented in class I MHC molecules. A new selection of cells bearing different antigens and/or cells without co-stimulating molecules is carried out. Finally, the selection of cells with higher indifferentiation levels is directly related to the increase of inhibiting factors, such as interleukin 10 and TGF β produced by the tumor. These substances induce the dendritic cells so that they become promoters of the specific suppressor cells. This phenomenon enables the development of the tolerance to tumor, which has the possibility of growing and spreading in absolute freedom. Those therapeutic approaches, based on immune system manipulation which ignore these dynamics of cell populations will fail, since a single way of action by the use of a specific technique leads to the before mentioned selection phenomena and its subsequent failure in results for a long time. The percentage of tumors which respond to the action of a single immunotherapy approach is of less that 20%, regardless the effectiveness and energy of this approach. Therefore, a combination of techniques which contemplate the described dynamics must be used in order to elicit the desired effect in the appropriate time.

### Immunotherapy

Although the host's immune system is often inadequate to control tumor growth, there are several indicators of the possible manipulation and improvement of the immune system in order to favor tumor eradication. Some of these are: the presence of identifiable tumor antigens in most of the tumor cells, the identification of detectable, though ineffective, host responses; and a better understanding of the mechanisms by which tumor cells reject the immune response. Recent technological breakthroughs have generated new potential for tumor antigen immunotherapy. Within these we find: techniques for the isolation of lymphocyte subpopulations, identification and purification of tumor antigens, development of antigen selected T cells, increase of immune responses by cytokines and the production of antibodies which target the surface of tumor antigen.

The Monoclonal antibodies (MAB) against tumor antigens either used exclusively or bound to toxins can control tumor growth.

The appearance of monoclonal antibodies suggested the possibility of targeting and destroying the tumors. Specific tumor antibodies of the right isotype could direct tumor cell lysis by NK cells and activate NK cells through their Fc receptors. In order to do this, a specific tumor antigen, which is a molecule of the cell membrane, should be found. After this, a mouse is immunized with the selected antigen. Then the mouse's spleen is removed and its tissue dissociated to obtain a lymphocyte cell suspension. The lymphocytes are then fused with cells from a myeloma that produces IgG. The obtained hybrid cell suspension is called hybridoma. It is cultured by its dilution on a 96-well culture plate. The fused cells are layered in such a way as to allow a few of them in each compartment. They are then left to grow and the supematant of each compartment is analyzed in order to determine which cell clones generated antibodies. Then, the IgG secreting clones are expanded and the produced antibody is analyzed to determine its specificity and effectiveness in recognizing different tumors of the same cell type but from different patients. After this, the selected clones are expanded. The antibodies which are to be used are extracted from the supernatant of these clones. If, by the use of molecular engineering, the Fc portion of the antibody is replaced by a similar one from human origin; the antigenicity of this molecule will decrease. These are called “humanized” antibodies.

The FDA has recently approved the use of a humanized monoclonal antibody, known as herceptin for the treatment of breast cancer. This antibody reacts to the receptor of growth factor HER-2/neu. This receptor is over expressed in almost one fourth of patients suffering from breast cancer. This over expression is responsible for a HER-2/neu induced antitumor response by T cells, although HER-2/neu has been related to a worse prognosis. The herceptin is believed to act by blocking interaction of the receptor and its natural ligand thus reducing the level of expression of the receptor. The effects of this antibody can increase when combined with conventional chemotherapy.

There is a second FDA approved antibody known as Rituximab which acts through the recognition of CD 20. This is used for the treatment of B cell non-Hodgkin lymphoma. The union and grouping of CD 20 shed a signal that induces lymphocyte apoptosis.

The monoclonal antibodies conjugated with emission radioisotopes have been used to visualize tumors in order to monitor tumor extension and provide diagnosis.

In the first informed successful tumor treatment with monoclonal antibodies, anti idiotype antibodies were used to target those B cells whose immunoglobulin expressed the corresponding idiotype. The first part of treatment generally leads to remission; but the tumor reappears in a mutant form which does not bind the antibody used in the initial treatment. This case represents a clear example of genetic instability, which allows the treatment elusion by tumor.

Other problems presented by the therapeutic use of tumor specific or tumor selective monoclonal antibodies are the inefficient killing of cells after monoclonal antibody union and the inefficient penetration of the antibody in the tumor mass. The first problem could be frequently avoided by binding a toxin to the antibody. This procedure generates a reagent called immunotoxin. The two toxins preferably indicated for this procedure are ricine chain A and Pseudomonas toxin. Both of these approaches require the internalization of the antibody so as to allow separation of the toxin molecule from the antibody molecule in the endocytic compartment; thus enabling penetration of the toxin chain and the subsequent killing of the cell.

Two other assays which use conjugated monoclonal antibodies imply the union of the antibody molecule and chemotherapy drugs, such as adriamicine or the union of this molecule and radioisotopes.

In the first case, the monoclonal antibody specificity through an antigen from the tumor cells surface concentrates the drug in its location. After internalization the drug is released in the endosomes and exercises its cytostatic or cytotoxic effect. The monoclonal antibodies bound to radioisotopes concentrate radioactive focus on the tumor location. Both of these approaches are advantageous as they kill neighboring tumor cells, since once the drug or radioactive emissions are released, they can affect cells which are adjacent to those united to antibody.

The CEA, carcinoembryo antigen, is an example of tumor antigen target of monoclonal antibodies. A recurrent colorectal cancer can be detected through a monoclonal antibody radioactively marked against CEA. This procedure is currently at a trial stage for the diagnosis and therapy of this neoplasm.

### Dendritic Cells

DC have been shown to be “Mother Nature's” antigen-presenting cells that naturally function to process and deliver foreign antigens and “danger” signals to lymph nodes for presentation to T cells and generation of a protective immune response. When the DC are activated and “matured,” they appear to be more potent for the process of T-cell stimulation. DC normally are resident in skin and other visceral organs, where they would encounter pathogens and other antigens; and their intradermal injection after they are boosted with antigens has been shown to induce regression of melanoma and colorectal cancer in early trials.

DC comes from the bone marrow. IL3, SCF, Fit3L, TNF and GMCSF influence its early differentiation. This last cytokine promotes the proliferation of the pre differentiated forms and favors the release of these cells to the blood stream. Nonetheless, DC are the most potent vehicles known for the generation of immune responses from naive T cells and are used in processing and delivery of cancer antigen-specific vaccines.

Therapeutic cancer vaccines based on dendritic cells (DC) loaded with tumor antigens have been of special interest because of the central role DC play in immunity. DC are found throughout the body, particularly in areas that can be portals of entry for infectious organisms. Numerous studies of animal models have shown that DC loaded with tumor antigens could protect against a tumor challenge and that DC-based immunizations could slow progression of previously implanted tumors. For example, mice immunized with dendritic cells loaded with antigens derived from the B16 melanoma cell line could prevent progression of implanted tumors.

In order to mimic the physiologic migration of DC to regional lymph nodes, DC was used by different administration routes: intravenously (IV), subcutaneously (SC), intradermally, intranodally, intralymphatically, and intratumorally. Administration of cytokines, along with DC, may increase the immune response induced by the immunizations. In this invention thymalfasin used as immunostimulant improved the clinical response to DC vaccination in non responder patients.

In general, most DC vaccine-based studies have followed this approximate scheme. Patients undergo a leukapheresis to generate the DC. Usually, a fraction of these DC are used fresh for the first immunization, while the remainder is cryopreserved for later use. The DC are loaded with the antigen and the loading strategy of interest prior to immunization, although in some studies the loading is performed prior to cryopreservation so that the DC vaccine is ready to use after thawing. The ideal interval or duration for immunization is unknown, but generally they are given every 1 to 3 weeks. DC loaded with irrelevant antigens are included as positive and negative controls for the immunizations. Then peripheral blood is drawn to monitor the induction of immune responses; but to perform the extensive immune analyses on the final product, a repeat leukapheresis could be performed. A variety of assays are now being used in clinical trials. In addition to measures of activity in vivo, it is possible to characterize the T-cell response in vitro by determining cytokine production, proliferation, or cytolytic activity of T cells in response to the immunizing antigen.

As the ongoing trial is general, DC vaccines have been well tolerated with minor toxicity. There are on going trials with other potential oncology vaccines (cell vaccines, Melacines vaccines, allogenix cell vaccines alone or with BCG, vaccinia oncolysate, cell free supernatant vaccines, genetic vaccinations, viral vector vaccines.)

Many attempts have been made to increase the immunogenicity of the oncology vaccination, they include: Keyhole limpet hemocyanin (KLH): is a protein made by a shelled sea creature found along the coast of California and Mexico known as a keyhole limpet. KLH is a large protein that causes an immune response and acts as a carrier for cancer cell antigens. Cancer antigens are often relatively small proteins that may be invisible to the immune system. KLH provides additional recognition sites for immune cells known as T-helper-cells and may increase activation of other immune cells known as cytotoxic T-lymphocytes (CTLs).

Bacillus Calmette Guerin (BCG): is an inactivated form of the tuberculosis bacterium routinely used for decades to vaccinate against TB. BCG is added to some cancer vaccines with the hope that it will boost the immune response to the vaccine antigen. It is not well understood why BCG may be especially effective for eliciting immune response. However, BCG has been used for years with other vaccines, including the vaccine for tuberculosis.

Interleukin-2 (IL-2): is a protein produced by the body's immune system that may boost the cancer-killing abilities of certain specialized immune system cells called natural killer cells. Although it can activate the immune system, many researchers believe IL-2 alone will not be enough to prevent cancer relapse. Several cancer vaccines use IL-2 to boost immune response to specific cancer antigens.

Granulocyte Monocyte-Colony Stimulating Factor (GM-CSF): is a protein that stimulates the proliferation of antigen-presenting cells.

QS21: is a plant extract that may improve the immune response when added to some vaccines.

These intend to enhance the biologic response to the cancer vaccines. Nobody had ever described the use of a broad immunostimulant drug, thymosin alpha 1 (thymalfasin), as an immunostimulator in combination with cancer vaccines. We saw that this agent modifies or increases the biological response to the dendritic vaccination (oncology vaccines). We use this immunostimulant drug in breast cancer patients that did not present a previous response to a dendritic cell vaccine with very good response.

Thymalfasin alpha 1 or Tα1, is a peptide that has been used for its immunomodulatory action and related therapeutic potential in several diseases, including chronic hepatitis B and C, acquired immunodeficiency syndrome (AIDS), primary immunodeficiency diseases, depressed response to vaccination, and cancer. The basis for its effectiveness in these conditions is primarily through modulation of immunological responsiveness. This drug has shown to have beneficial effects on numerous immune system parameters and to increase T-cell differentiation and maturation.

Thymalfasin alpha 1 was originally isolated as a natural substance from thymus tissue. It is a pure, synthetic amino-terminal acylated peptide of 28 amino acids (molecular weight 3108). Now, TA1 is produced by solid phase peptide synthesis.

Endogenous thymalfasin can be detected in serum, where levels measured in healthy adults by immunoassays are in the range of 0.1 to 1 ng/mL. The source and mechanisms of release and regulation of circulating thymalfasin are unknown. It is possible that thymalfasin has intracellular receptors, as it can fold into a structured helix in organic solvents and thus may cross the membrane unassisted.

Thymalfasin stimulates stem cells to produce increased numbers of mature T cells. The addition of thymalfasin to human CD34 stem cells in culture increased thymopoiesis, resulting in an increase in the number of total CD3 T cells and synthesis of interleukin-7 (IL-7), a cytokine critical for maturation of thymocytes. The increased predominant subpopulation was helper T cells (CD4).

Enhanced production of CD3, CD4, and CD8 cells in patients with chronic hepatitis B24 and cancer. Increased NK-cell activity in multiple animal models, normal human subjects, and HIV-infected patients.

Thymalfasin can increase production of IFNγ IL-2, IL-3, and the expression of the IL-2 receptor following activation by mitogens or antigens. This pattern of enhanced cytokine production, i.e., IFNγ and IL-2, demonstrates that thymalfasin promotes a Th1 type of immune response and induced a significant increase in the production of IL-2 as well as a decrease the Th2 cytokines IL-4 and IL-10.

The thymalfasin antagonizes dexamethasone-induced apoptosis in thymocytes in vitro in a dose-dependent fashion. The effects were most pronounced on CD4CD8 double positive immature T cells. Apoptosis of thymocytes stimulated by serum from tumor-bearing mice was also decreased by treatment with thymosin

Thymalfasin has been investigated in humans for treatment of infectious diseases (hepatitis B, hepatitis C, acquired immune deficiency syndrome), as a vaccine enhancement agent, and for several cancers, but nobody had used it as an immunomodulator with cancer vaccines.

This drug has shown efficacy in several animal cancer models and has been shown to improve immune function. Many cancer patients have depressed cellular immunity, and progression of some cancers appears to be related to impaired suppression of the tumors by the immune system.

The exact mechanism of action that can explain how thymalfasin can improve the clinical response to cancer vaccines is not completely understood. This action could be related with many of the mechanisms that the drug was shown and/or with others that are not known to date. It may be related with the cytokine polarization to Th1 and C1 reaction observed, which in turn develops an environment to induce DC to start effector rather than suppressor immune activities.

Thymalfasin is a safe drug and its potential side effects are significantly low.

As described herein, DC-TBH is an active immunotherapy treatment involving the periodical immunization of patients with autologous Dendritic Cells (DC) co-cultured with autologous tumor cells fused with activated autologous B cells (TBH).

TBH is used as a source of tumor antigens and DC are used as antigen presenting cells.

In preferred embodiments, the present invention presents several features which are advantageous for obtaining good therapeutic results in patients suffering from advanced neoplasic diseases.

Although it is advisable to obtain a great number of cells as in the case of dealing with a surgical piece, the number of tumor cells obtained through a fine needle biopsy is sufficient for the elaboration of TBH. In this way, the patient is prevented from going through any unnecessary surgical risks. On the other hand, and as it is mentioned below, the metastasis antigenicity seems to be different in every different organ. Therefore it is preferred to utilize a non-invasive method to obtain tumor cells from substantially every metastasis site of the patient.

B lymphocytes are cells which once activated become, due to their efficiency, the second most powerful type of antigen presenting cells in the immune system. On the other hand, if B cell cultures are stimulated with IL6 they may continue growing for at least 6 months. This IL6 sensitivity is transmitted to the TBH population after cell fusion.

Therefore TBH could be generated from a few tumor cells and maintained and expanded in vitro for several months, without losing its potential and antigenic diversity.

Once the DC are exposed to this hybrid, they capture substantially all the possible tumor antigens which are present in the natural state of the different neoplasic cell populations. These antigens are presented on the TBH surface together with a group of co-stimulating and adhesive molecules characteristic of activated B cells, which allow their utmost efficient capture and elaboration by the DC, even at low concentration levels.

The efficacy of the therapeutic action of the DC involving treatments appears to be directly related to the source these cells were obtained from.

According to a bibliographical compilation, approximately 68% of patients treated with DC obtained through mobilization of young and mature forms from the marrow bone presented reduction of the tumor mass of over 50%; whereas in the case of patients who were treated with DC generated in vitro by differentiation of CD34+ or circulating monocytes presented reduction of under 20%.

The DC used in the herein described exemplary protocol were obtained from the Buffy Coat of patients who were stimulated with GMCSF at low doses for five days. This enabled the collection of mature and immature forms and a low flow of CD34+. On the other hand, the in vitro culture with GMCSF and TNF for only three days and the absence of IL4 allows the differentiation of effector DC and prevents the differentiation of other possibly present cells forms, such as CD34+, CD14+ or monocytes.

No significant statistical differences were found between immune response and patient survival when the DC used for immunization have been obtained through sedimentation or by negative selection using an antibody cocktail which excludes CD34+ and CD14+.

### Exemplary Protocol

DC comes from the bone marrow. IL3, SCF, Fit3L, TNF and GMCSF influence its early differentiation. This last cytokine promotes the proliferation of the predifferentiated forms and favors the release of these cells to the blood stream.

The subcutaneous administration of GMCSF elicits an important passage of DC to blood.

After that, it is possible to isolate them in a therapeutically useful number in a blood sample obtained by apheresis and posterior negative selection, using for that purpose the StemSep™ kit for DC, provided by Stem Cell Technology, Vancouver, Canada. The GMCSF that we have used is human recombinant in E. Coli, produced by Cassara Laboratory from Argentina. The dose we have chosen is of 150 μg, daily administered in the evening (at approximately 7 pm) for five consecutive days. With this schedule of dose and administration, the effect is high in relation to the number of obtained DC, with a low increase of granulocytes and appearing of side effects. At that moment, the DC from bone marrow origin that pass to the blood stream have:
(1) The ability to pass through the capillary walls. They also have poor mobility.
(2) Great phagocytic ability, but poor Antigen Presentation Capacity.
(3) They are not defined as whether they induce effector or tolerance reaction.

They go into the tissue where they stay in alert and by cytokine action of the microenvironment, as well as for the phagocytic act, they differentiate to adult forms acquiring other characteristics:
(1) Their membrane receptors mutate and they acquire the ability of migrating from the tissues to the lymphatic capillary vessels and pass through them. They acquire great mobility, but loose their ability to pass the capillary walls.
(2) They loose their phagocytic ability, but increase their antigen presenting ability.
(3) They define themselves as inducers of regulator or effector immune reaction

### Treatment Description

Samples were obtained from the patients' different metastases. Through an apheresis and ulterior purification process performed in the laboratory, the patients' B cells were purified, and activated in vitro for 48 hours through adding IL 4 and IL 6.

Finally, the patients were immunized with the activated B cells hybrid itself, or a B cell hybrid cocultured with the patients' Dendritic Cells. This immunization was applied into a healthy lymph node once every three weeks. At the same time, the patients received 1.6 mg thymalfasin subcutaneously in the evenings (within between 7:00 pm and 9:00 pm) every 3 days during the time of immunization and for the following six months after the vaccination protocol was completed. This vaccination plan may, for example, involve from 4 to 10 doses, although these numbers are not exclusive.

B cells are obtained from the Buffy Coat of the patient's peripheral blood through hemapheresis. The product from apheresis is then seeded on a Ficoll-Hypaque gradient. The mononuclear cell ring obtained in the superior interphase is the source of B cells, which are isolated by negative selection using a commercial kit supplied by Stem Cell Technology from Vancouver, Canada. B cells are cultured in a serum free medium enriched with IL4 and IL6.

A tumor sample is obtained through a surgical or needle biopsy. Simultaneous cytological confirmation of the extracted material is performed in either case. The tumor sample is mechanically dissociated. The single cell suspension obtained is cultured in a serum free medium enriched with human albumin, insulin, and epidermal growth factor.

The activated lymphocytes and the isolated tumor cells are then fused by the use of a polyethylenglycol solution. The formation of TBH cells is controlled through immune double stain with anti CD20 as a B cell marker, and anti cytokeratin or anti vimentine according to tumor cells source. The hybrids are then cultured in a serum free medium enriched with insulin, epidermal growth factor, and IL6.

The autologous DC are obtained through hemapheresis after being mobilized from the Bone marrow. Mobilization is performed by stimulating the patient with GMCSF for 5 days. The Buffy Coat corresponding to the process of two blood volumes is collected via apheresis on the sixth day.

A mixed population of immature and differentiate DC is concentrated from the patient's Buffy Coat. This concentration and purification step may be carried out either by a differential adhesion technique or by negative selection. In the former, mononuclear cells are layered on a tissue culture flask, and four hours later the supernatant is gently disposed. The adherent cells are then cultured in the appropriate tissue culture medium described below. In the negative selection method, mononuclear cells are incubated with a mixture of 8 monoclonal antibodies (MAB) against: CD 3, CD14, CD16, CD19, CD 34, CD56, CD66b and Glycophorin A. Each monoclonal antibody is conjugated with an immune magnetic bead. The marked cell suspension is purified by passing through a magnetic field. Marked cells are retained and non marked cells are collected in a sterile tube. The obtained non marked cell suspension is composed of 50% (40-60%) immature and mature DC suspension.

The autologous enriched DC suspension is co-cultured with autologous TBH for three days in a serum free medium enriched with human albumin, GMCSFrh, and TNFrh.

The DC are washed, concentrated and injected in one of the patient's healthy lymph nodes after their culture for 72 hours, followed by the corresponding safety, purity, and potency tests.

This procedure presents several features which are advantageous for the possibility of obtaining good therapeutic results in patients suffering from advanced neoplasic diseases.

Although it is an advantage to obtain a great number of cells as in the case of dealing with a surgical piece, the number of tumor cells obtained through a fine needle biopsy is sufficient for the elaboration of TBH. The metastasis antigenicity seems to be different in every different organ. Therefore it is very important to count on a non-invasive method to obtain tumor cells from every metastasis site of the patient.

B lymphocytes are cells which once activated become, the second most powerful type of antigen presenting cells in the immune system. On the other hand, if

B cell cultures are stimulated with IL6 they may continue growing for at least 6 months. This IL6 sensitivity is transmitted to the TBH population after cell fusion.

Therefore TBH could be generated from a few tumor cells and maintained and expanded in vitro for several months, without losing its potential and antigenic diversity.

Once the DC are exposed to this hybrid, they capture substantially all the possible tumor antigens which are present in the natural state of the different neoplasic cell populations. These antigens are presented on the TBH surface together with a group of co-stimulating and adhesive molecules characteristic of activated B cells, which allow their utmost efficient capture and elaboration by the DC, even at low concentration levels.

Since TBH is present in the DC as from the beginning of the in vitro maturity and activation processes, it allows the incorporation of the tumor antigens during the short period in which DC are able to carry out this process. Soon after the tumor antigens are endocytated, the DC reach the maximum level of efficiency in their ability to process and present antigens. They also develop the ability to migrate from the blood vessels to the tissues.

Thus the intra lymph node injection appears to be more efficient than the blood transfusion of the DC vaccine.

However, DC obtained through mobilization from the bone marrow results in a low number of cells obtained in a single procedure. When these DC are stimulated by the use of antigens which represent the whole tumor, such as tumor lysated from a surgical piece, or by a hybrid out of tumor cells and DC, the samples may be separated in different portions to achieve efficiency through time.

From a clinical evolution point of view, only some patients have a spontaneous good evolution with oncology vaccination alone. It is known that the patients with advanced breast cancer resistant to chemo, radio, and hormonal therapy have low survival rates.

A protocol of autologous dendritic cell vaccine (DCV) has been developed that may improve patient outcomes.

Thymalfasin (ZADAXIN®) has been shown to enhance Th1 response, which is associated with tumor regression. The following study was conducted to evaluate dendritic cell immunization and whether thymalfasin has a positive effect on the outcomes of advanced breast cancer patients who do not respond to vaccine therapy alone.

The invention is illustrated by the following example, which is not intended to be limiting.

### EXAMPLE

Eighteen patients with advanced breast cancer resistant to chemo, radio and hormonotherapy were treated.

All the patients were female with Breast Cancer class 4 (with metastases).

The age rank was between 39 to 71 years.

The patients were treated with dendritic cell vaccine (according protocol: Annals of Oncology 2004-Vol 15. Supp. 3 Abs: iii40-Dendritic Cell Vaccine for Metastases Breast Cancer).

After the second vaccination course, the cellular immunization were measured, if this is >=20 ULPI (Linfocitic Proliferative Index) continued with the vaccination.

If the response was <20 ULPI, the patients were divided in 2 groups (randomized) of 5 and 7 patients group. The 5 patients group received Dendritic Cells

Vaccine plus thymalfasin (1.6 mg/tw during 6 months). The other 7 patients group, did not receive immunostimulation, and received the programmed dendritic vaccination course.

A critical point for the success of this immunotherapy regimen was the early immune response that the patients had after the second DC vaccine.

The patient immune response was measured using a well known in-vitro test named Lymphocyte Proliferation Assay. Briefly, mononuclear cells from the patient were purified and mixed with a suspension of the patient tumor cells at ratio 10:1 (3000 Lympho-monocytes vs 300 tumor cells). The mix cell suspension was seeded in a multi well plate and incubated at 37° C. After 96 hr the cells were harvested and counted by an automatic haemocytometer.

If the number of mononuclear cells counted was higher than 20000 cells (Lymphocyte Proliferation Index-LPI of 20 U) the patient had a good outcome with effective tumor response and long survival. Opposite if after this mix cellular culture the mononuclear cells had not reached this number, the patient had a poor response, and the survival was significantly shorter than the immune responder patients.

Thymalfasin treatment is an important immune enhancer of Th1 response, which is the one involved on the tumor rejection.

Five consecutive advanced breast cancer patients of age rank between 39 to 71 years of age that after the second DC immunization had a LPI lower than 20 U were treated with thymalfasin (1.6 mg/twice a week during 6 months) plus 4 additional courses of Dendritic Cells Vaccines. Thymalfasin was able to improve the LPI and the majority of the treated patients showed an effective tumor response.

The clinical response and the survival data originated on total 18 metastatic breast patients treated were collected and, in order to perform a case serial statistic analysis we divided the total population in three different groups.

In Group 1 (n=7), patients received 6-dendritic cell immunizations (1 every 3 weeks) and had a lymphocyte proliferation index (LPI) >20 U (immune response) after the second vaccine.

In Group 2 (n=6), patients received 6 dendritic cell immunizations and had an LPI <20 U (non immune response) after the second vaccine.

In Group 3 (n=5), patients received 6 dendritic cell immunizations with an LPI <20 U (non immune response) after the second vaccine and they received thymalfasin (1.6 mg/twice a week).

Immune response was measured by lymphocyte proliferation assay. The results observed was at 6 months, tumor reduction of >50% in 100% of patients in Group 1 (responders), 50% in Group 2 (non responders), and 80% in Group 3 (non responders treated with thymalfasin).

Patient survival at 12 months was 57% in Group 1, 0% in Group 2, and 80% in Group 3.

We can see that an immune response (LPI >20 U) to DCV therapy after the second vaccination was associated with reduction in tumor size and longer patient survival. Treatment with thymalfasin had a positive effect on advanced breast cancer patients who did not respond to dendritic cell immunization. Patient survival was higher in the thymalfasin-treated group compared with immune responders and non immune responders who did not receive thymalfasin. (See Table 1).

Table 1 shows the Clinical Response at Month 6 and Patient Survival Rates at 12 Months.

TABLE 1
Clinical Response at Month 6 and Patient
Survival Rates at 12 Months

| | | Patients with | Patient |
|---|---|---|---|
| Treatment Arm | n | Tumor Reduction >50% | Survival |
| Group 1 (RESPONDERS) | 7 | 100% | 57% |
| Group 2 (NON RESPONDERS) | 6 | 50% | 0% |
| Group 3 (NON RESPONDERS | 5 | 80% | 80% |
| TREATED WITH | | | |
| THYMALFASIN) | | | |

A retrospective observation of 18 patients with metastatic breast cancer was conducted.
Group 1 (n = 7), patients received 6 dendritic cell immunizations (1 every 3 weeks) and had a lymphocyte proliferation index (LPI) >20 U (immune response) after the second vaccine.
Group 2 (n = 6), patients received 6 dendritic cell immunizations and had an LPI <20 U (non immune response) after the second vaccine.
Group 3 (n = 5), patients received 6 dendritic cell immunizations with an LPI <20 U (non immune response) after the second vaccine and they received thymalfasin (1.6 mg/twice a week).
Results: At 6 months, tumor reduction of >50% was seen in 100% of patients in Group 1 (responders), 50% in Group 2 (non responders), and 80% in Group 3 (non responders treated with thymalfasin). Patient survival at 12 months was 57% in Group 1, 0% in Group 2, and 80% in Group 3.

The use of this immunostimulant drug is not restrictive to patients with breast cancer treated with dendritic cell vaccines; instead it can improve the evolution and clinical results of the dendritic cell vaccines in patients with other types of cancer. Thymalfasin can also improve the clinical outcome with other kinds of immunology vaccines.

1. A pharmaceutical combination for treatment of cancer in a subject and for enhancing cancer vaccine effectiveness in the subject, comprising:
a) an immune response-triggering cancer vaccine capable of eliciting an immune system response in said subject; and
b) a vaccine effectiveness-enhancing amount of an alpha thymosin peptide, which enhances said immune system response in said subject;
c) wherein said cancer vaccine and said alpha thymosin peptide can be administered separately or together.

2. A pharmaceutical combination of claim 1, wherein said subject is human, and said vaccine is a dendritic cell vaccine.

3. The pharmaceutical combination of claim 1, wherein said vaccine is in an amount of from about 1×10⁻⁹ g to about 1×10⁻³ g, and said alpha thymosin peptide is in an amount of about 0.1-20 mg.

4. The pharmaceutical combination of claim 1, wherein said vaccine is in an amount of from about 1×10⁻⁸ g to about 1×10⁻⁴ g, and said alpha thymosin peptide is in an amount of about 0.5-10 mg.

5. The pharmaceutical combination of claim 4 wherein said alpha thymosin peptide is TA1, and the amount of said TA1 is about 1.6-3.2 mg.

6. The pharmaceutical combination of claim 1 wherein said cancer is breast cancer.

7. The pharmaceutical combination of claim 1 wherein said cancer is selected from the group consisting of primary melanoma, metastatic melanoma, adenocarcinoma, squamous cell carcinoma, adenosquamous cell carcinoma, thymoma, lymphoma, sarcoma, lung cancer, liver cancer, non-Hodgkins lymphoma, Hodgkins lymphoma, leukemias, uterine cancer, prostate cancer, ovarian cancer, pancreatic cancer, colon cancer, multiple myeloma, neuroblastoma, NPC, bladder cancer, cervical cancer, kidney cancer, brain cancer, bone cancer, uterine cancer, stomach cancer and rectal cancer.

8. A method of treating cancer in a subject comprising administering to the subject a pharmaceutical combination according to claim 1 for enhancing cancer vaccine effectiveness in said subject, said pharmaceutical combination comprising:
a) an immune response-triggering cancer vaccine capable of eliciting an immune system response in said subject; and
b) a vaccine effectiveness-enhancing amount of an alpha thymosin peptide, which enhances said immune system response in said subject;
c) wherein said cancer vaccine and said alpha thymosin peptide can be administered separately or together;
said method comprising administering said immune response-trigging cancer vaccine to said subject in conjunction with administering said alpha thymosin peptide to said subject, wherein said vaccine and said alpha thymosin peptide are administered to said subject separately or together.

9. The method of claim 8, wherein said subject is human, and said vaccine is a dendritic cell vaccine.

10. The method of claim 8, wherein said vaccine is in an amount of from about 1×10⁻⁹ g to about 1×10⁻³ g, and said alpha thymosin peptide is administered in an amount of about 0.1-20 mg.

11. The method of claim 8, wherein said vaccine is administered in an amount of from about 1×10⁻⁸ g to about 1×10⁻⁴ g, and said alpha thymosin peptide is in an amount of from about 0.5-10 mg.

12. The method of claim 11 wherein said alpha thymosin peptide is TA1, and said TA1 is administered in an amount of about 1.6-3.2 mg.

13. The method of claim 12, wherein said TA1 is administered substantially concurrently with administration of said vaccine.

14. The method of claim 12, wherein said vaccine and said TA1 are administered by injection.

15. The method of claim 8 wherein said combination is administered to said subject a plurality of times.

16. The method of claim 15 wherein said vaccine is administered to said subject 4-10 times during a course of administration.

17. The method of claim 16 wherein said vaccine is administered to said subject every third week during said course of administration.

18. The method of claim 17 wherein said alpha thymosin peptide is TA1, and wherein said TA1 is administered twice weekly during said course of administration.

19. The method of claim 18 wherein said course of administration is about six months.
